(19) United States
(12) Patent Application Publication
(10) Pub. No.: US 2014/0114177 A1
(43) Pub. Date: Apr. 24, 2014

(54) SYSTEM AND METHOD FOR MAGNETIC RESONANCE ELASTOGRAPHY OF THE BREAST

(71) Applicants: Jun Chen, Rochester, MN (US); Kevin J. Glaser, Rochester, MN (US); Richard L. Ehman, Rochester, MN (US); Jennifer L. Kugel, Rochester, MN (US)

(72) Inventors: Jun Chen, Rochester, MN (US); Kevin J. Glaser, Rochester, MN (US); Richard L. Ehman, Rochester, MN (US); Jennifer L. Kugel, Rochester, MN (US)

(21) Appl. No.: 14/112,324

(22) PCT Filed: Apr. 17, 2012
(86) PCT No.: PCT/US12/33932

§ 371 Date: Jan. 2, 2014

Related U.S. Application Data
(60) Provisional application No. 61/478,313, filed on Apr. 22, 2011.

(51) Int. Cl. G01R 33/563; A61B 5/055

(52) U.S. Cl. CPC G01R 33/56358; A61B 5/0555
USPC 600/415; 600/421

(57) ABSTRACT

A system and method for performing magnetic resonance elastography (MRE] of a patient's breasts is provided. An MRE driver configured to be placed on the sternum of the patient is used to impart mechanical energy to the sternum, which in turn generates shear waves in at least one of the patient's breasts. Such a driver is amenable to use with standard breast radio frequency (RF] coils without the need for modification of the existing breast RF coil hardware.

### CROSS-REFERENCE TO RELATED APPLICATIONS

This application claims the benefit of U.S. Provisional Patent Application Ser. No. 61/478,313, filed on Apr. 22, 2011, and entitled “SYSTEM AND METHOD FOR MAGNETIC RESONANCE ELASTOGRAPHY OF THE BREAST.”

### STATEMENT REGARDING FEDERALLY SPONSORED RESEARCH

This invention was made with government support under EB001981 awarded by the National Institutes of Health. The government has certain rights in the invention.

### BACKGROUND OF THE INVENTION

The field of the invention is systems and methods for magnetic resonance imaging (“MRI”). More particularly, the invention relates to systems and methods for magnet resonance elastography (“MRE”).

Breast cancer is one of the most commonly diagnosed life-threatening diseases in American women. In the clinical application, x-ray mammography and contrast-enhanced MRI (“CE-MRI”) have been routinely used for screening and detecting breast cancer; however, both of these techniques have high sensitivity but low specificity.

Elastography, which provides a measurement of how stiff a tissue is, has shown promise in detecting and characterizing diseased tissue. Palpation and breast self-examination have been used to subjectively feel the tissue stiffness change in breasts in order to detect suspicious pathological breast tissue. Previous studies have shown that malignant tumor samples are significantly stiffer than benign tumor samples. A recent study has also shown that breast MRE, a technique for measuring the stiffness of breast tissue, can improve the specificity by as much as twenty percent, while maintaining sensitivity near one-hundred percent when compared with CE-MRI alone.

Breast MRE uses a driver to transmit mechanical waves to the breasts, while acquiring images that are influenced by these mechanical waves. Using an inversion algorithm, the mechanical properties of the breasts can be calculated. The design of a breast MRE driver is important because all of the MRE processing is based on having a detectable mechanical wave generated in the tissue of interest by the driver. Breast driver design is challenging because by their very nature, the breasts have fat content that attenuates the penetration of mechanical waves into the breasts. Moreover, different patients will have differently sized breasts. Breast MRE driver design is also complicated because commercial breast radio frequency (“RF”) coils and narrow MRI bores have limited space for positioning and adjusting the driver. Usually, RF breast coils require modifications to accommodate the positioning of a driver for breast MRE. In addition, the positioning of the driver could interfere with the MRI-guided breast biopsy.

Notwithstanding the above challenges, different breast drivers have been developed for breast MRE scans. These drivers were put inside the RF breast coils such that the driver makes direct contact with the breasts, either on the right-left or the anterior-posterior sides of breasts. These previously reported breast drivers all have the same limitation that they must be in direct contact with the breast in order to transmit mechanical waves into the breast. In addition to the foregoing challenges with breast MRE driver design, those drivers that make direct contact with the breast have further disadvantages. These disadvantages include adding tension and changing the shape of the breasts, which are factors that affect the measure of mechanical properties of the breast; and providing undesirable mechanical coupling between the driver and the breast.

In light of the foregoing, it would be advantageous to provide an MRE driver system that is suitable for bilateral breast MRE that does not directly contact the breasts and that is compatible with existing RF breast coils. Such a driver should minimize interference with current clinical breast MRI and MRI-guided breast biopsy setups while keeping mechanical wave SNR high enough for MRE processing.

### SUMMARY OF THE INVENTION

The present invention overcomes the aforementioned drawbacks by providing a system and method for performing magnetic resonance elastography (“MRE”) of the breast using an MRE driver that does not directly contact the subject's breasts. Generally, the MRE driver is configured to direct mechanical energy into the subject's sternum, which is then converted into tissue motion in the subject's breasts. Such an MRE driver is compatible with existing radio frequency (“RF”) breast coils.

Because the MRE driver directly contacts the subject's sternum and not their breasts, the MRE driver has the following advantages. The MRE driver does not require additional space to be positioned between the subject and existing breast RF coils. The MRE driver does not add tension or otherwise change the shape of the subject's breasts. The MRE driver is not affected by the different sizes of different subjects' breasts. The MRE driver does not interfere with MRI-guided breast biopsies.

It is an aspect of the invention to provide an acoustic driver for applying acoustic energy to a subject during a magnetic resonance elastography (“MRE”) examination. The acoustic driver includes a cavity that is configured to receive acoustic energy and a flexible enclosure surrounding the cavity. The flexible enclosure is sized for placement adjacent a subject's sternum. The flexible enclosure includes an intake extending through the flexible enclosure and into the cavity. This intake is configured to be coupled to a tube in order to receive acoustic energy for delivery into the cavity.

It is another aspect of the invention to provide a method for performing magnetic resonance elastography (“MRE”) of a subject's breast using an MRI system. The method includes positioning an MRE driver on the subject's sternum and operating the MRE driver so that mechanical energy is imparted to the sternum such that shear waves are produced in at least one of the subject's breasts. By way of example, the MRE driver is positioned such that it does not contact either of the subject's breasts. The MRI system is then directed to acquire image data of the subject while the shear waves are produced in the at least one of the subject's breasts. Images of the subject that depict propagation of the shear waves through the at least one of the subject's breasts are reconstructed from the acquired image data, and mechanical properties of the at least one of the subject's breasts are calculated from these images.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a block diagram of an exemplary magnetic resonance imaging (“MRI”) system that employs the present invention;

FIG. 2 is a pictorial representation of an MRI system the employs an implementation of the present invention;

FIG. 3 is a cross-sectional view of one configuration of an acoustic driver suitable for performing MRE of the breast, the cross-sectional view showing the acoustic driver positioned on a subject and flexed accordingly;

FIG. 4 a cross-sectional view of another configuration of an acoustic driver suitable for performing MRE of the breast, the cross-sectional view showing the acoustic driver positioned on a subject and flexed accordingly;

FIG. 5 is a pulse sequence diagram of an example of a pulse sequence for acquiring MRE data from a subject; and

FIG. 6 is a pulse sequence diagram of an example of another pulse sequence for acquiring MRE data from a subject

### DETAILED DESCRIPTION OF THE INVENTION

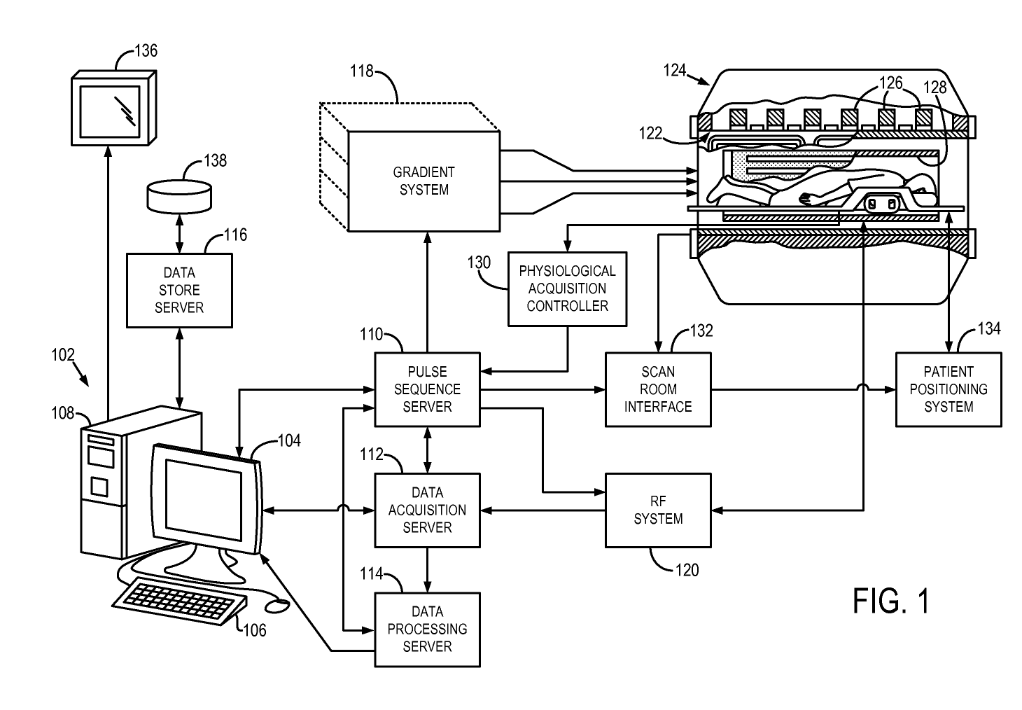

A system and method for performing magnetic resonance elastography (“MRE”) of the breast, including an MRE driver that is amenable for breast MRE, are provided. Referring to FIG. 1, an exemplary magnetic resonance imaging (“MRI”) system 100 for use with embodiments of the present invention is illustrated. The MRI system 100 includes a workstation 102 having a display 104 and a keyboard 106. The workstation 102 includes a processor 108, such as a commercially available programmable machine running a commercially available operating system. The workstation 102 provides the operator interface that enables scan prescriptions to be entered into the MRI system 100. The workstation 102 is coupled to four servers: a pulse sequence server 110; a data acquisition server 112; a data processing server 114, and a data store server 116. The workstation 102 and each server 110, 112, 114 and 116 are connected to communicate with each other.

The pulse sequence server 110 functions in response to instructions downloaded from the workstation 102 to operate a gradient system 118 and a radiofrequency (“RF”) system 120. Gradient waveforms necessary to perform the prescribed scan are produced and applied to the gradient system 118, which excites gradient coils in an assembly 122 to produce the magnetic field gradients Gₓ, G_{y}, and G_{z} used for position encoding MR signals. The gradient coil assembly 122 forms part of a magnet assembly 124 that includes a polarizing magnet 126 and a whole-body RF coil 128.

RF excitation waveforms are applied to the RF coil 128, or a separate local coil (not shown in FIG. 1), by the RF system 120 to perform the prescribed magnetic resonance pulse sequence. Responsive MR signals detected by the RF coil 128, or a separate local coil (not shown in FIG. 1), are received by the RF system 120, amplified, demodulated, filtered, and digitized under direction of commands produced by the pulse sequence server 110. The RF system 120 includes an RF transmitter for producing a wide variety of RF pulses used in MR pulse sequences. The RF transmitter is responsive to the scan prescription and direction from the pulse sequence server 110 to produce RF pulses of the desired frequency, phase, and pulse amplitude waveform. The generated RF pulses may be applied to the whole body RF coil 128 or to one or more local coils or coil arrays (not shown in FIG. 1).

The RF system 120 also includes one or more RF receiver channels. Each RF receiver channel includes an RF amplifier that amplifies the MR signal received by the coil 128 to which it is connected, and a detector that detects and digitizes the I and Q quadrature components of the received MR signal. The magnitude of the received MR signal may thus be determined at any sampled point by the square root of the sum of the squares of the I and Q components:

M=\sqrt{I²+Q²} (1);

and the phase of the received MR signal may also be determined:

$$φ = {tan}^{- 1} ( \frac{Q}{I} ) . \qquad ( 2 )$$

The pulse sequence server 110 also optionally receives patient data from a physiological acquisition controller 130. The controller 130 receives signals from a number of different sensors connected to the patient, such as electrocardiograph (“ECG”) signals from electrodes, or respiratory signals from a bellows or other respiratory monitoring device. Such signals are typically used by the pulse sequence server 110 to synchronize, or “gate,” the performance of the scan with the subject's heart beat or respiration.

The pulse sequence server 110 also connects to a scan room interface circuit 132 that receives signals from various sensors associated with the condition of the patient and the magnet system. It is also through the scan room interface circuit 132 that a patient positioning system 134 receives commands to move the patient to desired positions during the scan.

The digitized MR signal samples produced by the RF system 120 are received by the data acquisition server 112. The data acquisition server 112 operates in response to instructions downloaded from the workstation 102 to receive the real-time MR data and provide buffer storage, such that no data is lost by data overrun. In some scans, the data acquisition server 112 does little more than pass the acquired MR data to the data processor server 114. However, in scans that require information derived from acquired MR data to control the further performance of the scan, the data acquisition server 112 is programmed to produce such information and convey it to the pulse sequence server 110. For example, during prescans, MR data is acquired and used to calibrate the pulse sequence performed by the pulse sequence server 110. Also, navigator signals may be acquired during a scan and used to adjust the operating parameters of the RF system 120 or the gradient system 118, or to control the view order in which k-space is sampled.

The data processing server 114 receives MR data from the data acquisition server 112 and processes it in accordance with instructions downloaded from the workstation 102. Such processing may include, for example: Fourier transformation of raw k-space MR data to produce two or three-dimensional images; the application of filters to a reconstructed image; the performance of a backprojection image reconstruction of acquired MR data; the generation of functional MR images; and the calculation of motion or flow images.

Images reconstructed by the data processing server 114 are conveyed back to the workstation 102 where they are stored. Real-time images are stored in a data base memory cache (not shown in FIG. 1), from which they may be output to operator display 112 or a display 136 that is located near the magnet assembly 124 for use by attending physicians. Batch mode images or selected real time images are stored in a host database on disc storage 138. When such images have been reconstructed and transferred to storage, the data processing server 114 notifies the data store server 116 on the workstation 102. The workstation 102 may be used by an operator to archive the images, produce films, or send the images via a network to other facilities.

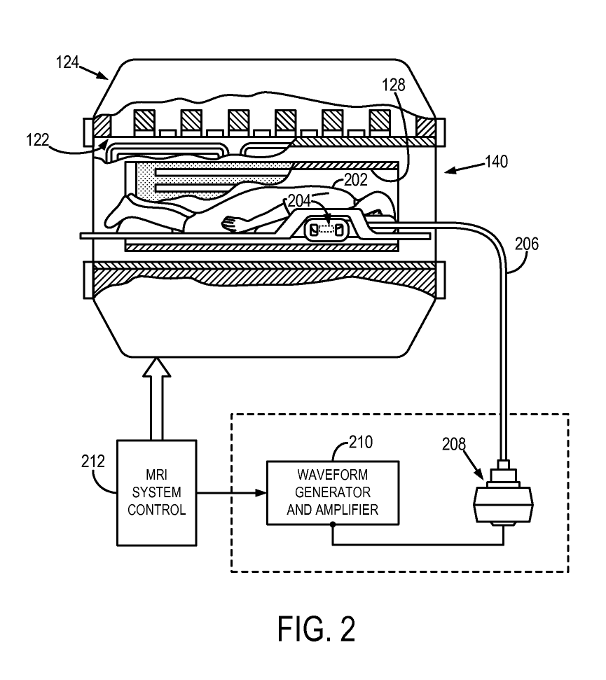

Referring now to FIG. 2, the MRE driver system of the present invention is a passive driver system that may be placed on a subject 202 and energized to produce an oscillating, or vibratory, stress. The MRE driver system includes a passive driver 204 positioned over a region-of-interest, such as the sternum, in the subject 202 and connected by means of a tube 206 to a remotely located active acoustic driver 208. The active driver 208 is remote from the bore 140 of the magnet assembly 124 in the sense that it is positioned away from the strong magnetic fields produced by the magnet assembly 124 where its operation is not impeded by those fields, and where its operation will not perturb the magnetic fields of the MRI system 100. The active driver 208 is electrically driven by a waveform generator and amplifier 210, which in turn is controlled by the pulse sequence server 110, which forms a part of the MRI system control 212. The MRI system control 212 directs the MRI system 100 to perform an MRE scan by driving the RF coil 128 and the gradient coils 122 in the magnet assembly 124 to perform a series of pulse sequences, while enabling the waveform generator 210 to apply an oscillatory stress to the subject 202 at the proper moment during each pulse sequence, as described in U.S. Pat. No. 5,592,085, which is herein incorporated by reference in its entirety. The active driver 208 and the waveform generator and amplifier 210 may be housed together in a manually portable unit, denoted by dashed line 214. Examples of active acoustic drivers 208 are disclosed in U.S. Pat. Nos. 7,034,534 and 7,307,423; and in U.S. Patent Application Publications No. US2009/0299168 and US2010/0005892.

The passive driver 204 is preferably positioned on the middle part, or bridge, of a standard breast radio frequency (“RF”) coil, such as the Liberty 9000 eight-channel breast coil (USA Instruments, Inc., Aurora, Ohio). During an MRE procedure, the patient is positioned feet first in the prone position on the coil with the driver 204 in contact with the patient's sternum.

The tube 206 may be made of a material that is flexible, yet inelastic. The flexibility enables it to be fed along a winding path between the subject 202 in the magnet 124 and the remote site of the active driver 208. In one configuration, the tube 206 has an inner diameter of one inch. The tube 206 may be composed of a clear vinyl material sold under the trademark TYGON—a registered trademark of Norton Company of Worchester, Mass.—and may have a wall thickness of approximately one-eighth inch. Alternatively, the tube 206 may include a polyvinyl chloride (“PVC”) tube with a reinforced wall having an inside diameter of approximately three-quarters of an inch. The tube 206 is inelastic such that it does not expand in response to the variations in air pressure caused by the acoustic energy it conveys. As a result, the acoustic energy is efficiently conveyed from the active driver 208 to the passive driver 204.

Using the above-described MRE driver system, the physical properties of tissue, such as breast tissue, can be measured using MRE by applying a stress to the subject 202 and observing the resulting strain. By measuring the resulting strain, elastic properties of the tissue, such as Young's modulus, Poisson's ratio, shear modulus, and bulk modulus can be calculated. By applying the stress in all three dimensions and measuring the resulting strain, the elastic properties of the tissue can be defined.

By observing the rate at which the strain decreases as a function of distance from the stress producing source, the attenuation of the strain wave can be estimated. From this, the viscous properties of the gyromagnetic medium may be estimated. The dispersion characteristics of the medium can be estimated by observing the speed and attenuation of the strain waves as a function of their frequency. Dispersion is potentially a very important parameter for characterizing tissues in medical imaging applications.

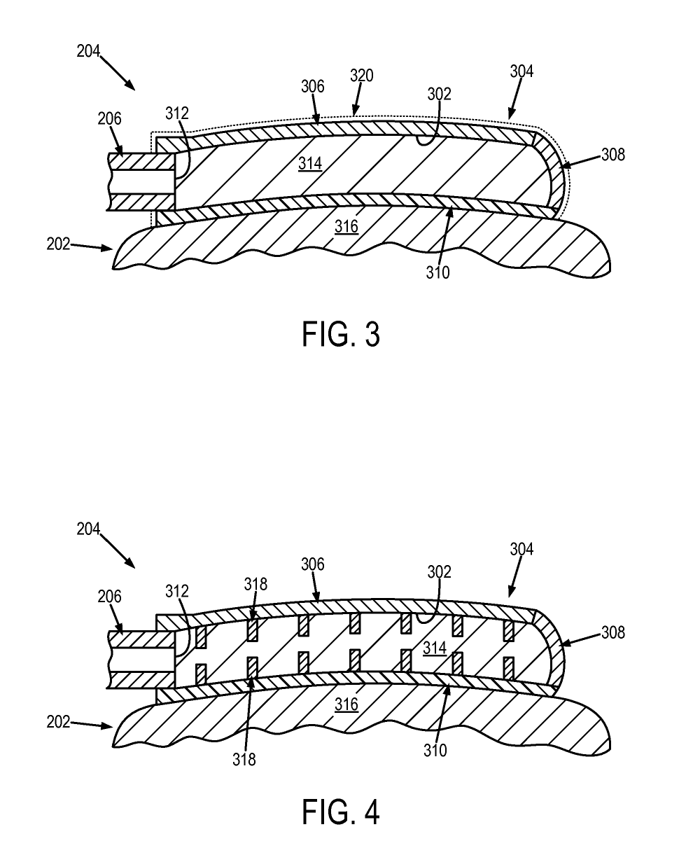

Referring to FIG. 3, an example of a passive acoustic driver 204 suitable for practicing the present invention is illustrated. The passive driver 204 includes a thin chamber 302 defined by an enclosure 304. The enclosure 304 is defined by an end wall 306 opposed by a flexible membrane 308. Side walls 310 extend from the end wall 306 to the flexible membrane 308 to define the chamber 302. An intake 312 is formed in one of the side walls 310 and provides a coupling between the tube 206 and the enclosure 304 such that the interior of the tube 206 is in fluid communication with the chamber 302. The enclosure 304 may be formed from a flexible materials, such as woven fabric; polycarbonate plastic; polystyrene foam, such as Styrofoam; foam rubber; a non-stretching material mesh, and the like. Each of the end wall 306, the flexible membrane 308, and the side walls 310 may be composed of the same or similar material. The chamber 302 is preferably filled with a highly porous, yet flexible fill material 314, such as a polyfiber material or loose, woven fabric.

The material used for the enclosure 304 may generally be any material that is flexible, but, preferably, the material is not stretchable and does not fold onto itself easily. In one configuration, the material has a built-in two-dimensional mesh of thread. This kind of material allows for the driver to conform to the subject and for motion to be imparted to the subject repeatedly, reliably, and efficiently, without the driver 204 undesirably deforming upon receiving the acoustic pressure waves from the active driver 208, which would result in inefficiently imparting vibrational energy to the patient 202. For sterilization purposes, a disposable cover 320 may be disposed about the flexible enclosure 304. Examples of such disposable covers 320 include disposable cloths or disposable films. Also for sterilization purposes, the passive driver 204 itself may be configured such that it is disposable. In this instance, the passive driver 204 would be discarded after it is used and a new passive driver 204 would be supplied for each new subject.

The fill material 314 that fills the chamber 302 may be any material that can support the end wall 306, side walls 308, and flexible membrane 310 that form the enclosure 304 and can keep those surfaces separated. This fill material 314 should also be porous to facilitate free air flow inside the driver 204. The fill material 314 maintains an appropriate spacing between the patient 202 and the end wall 306, and does not impede the pressure waves traveling through the fill material 314. By way of example, the fill material 314 maintains an appropriate spacing between the end wall 306 and the patient's sternum.

The flexible membrane 310 is placed against the skin 316 of the patient 202 and, along with the entire passive driver 204, conforms to the shape of the patient 202. By way of example, the flexible membrane 310 is placed against the skin 316 adjacent the patient's sternum. The membrane 310 vibrates in response to acoustic energy received by the passive driver 204 through the tube 206. In the foregoing example, the vibrations apply an oscillating stress to the patient's sternum, which is conveyed into the breast tissue as shear waves.

In one example configuration of the passive driver 204, which may be used for MRE of the breast, the enclosure 304 includes a small flexible strip constructed of an inelastic material, such as a rubber sheet, that is wrapped around a fill material 314 that is a porous, springy foam. Acoustic pressure is provided to the passive driver 204 by way of the active driver 208 located outside of the MRI scan room. By way of example, harmonic acoustic pressure oscillating at 60 Hertz is provided to the passive driver 204. The acoustic pressure is provided to the passive driver 204 from the active driver 208 by way of the tube 206 through the intake 312 and into the chamber 302 of the passive driver 204. The flexible strip that forms the enclosure 304 is sized to be placed on a patient's sternum. For example, the flexible strip may be 6.5×17×0.8 centimeters or it may be 3.5×20×0.8 centimeters. It is noted that the width of the passive driver 204 may impact the efficacy of the MRE procedure depending on characteristics of the patient 202, such as their size. For example, a wider driver 204 may contact and add pressure to the medial edge of the breast in some patients. In these instances, a narrower driver 204 will reduce the negative effects that such contact may produce. Although the driver 204 is mainly coupled to the sternum, the driver 204 generates extensive shear wave motion in both breasts.

Referring now to FIG. 4, another configuration of an acoustic driver suitable for MRE of the breast is illustrated. In this configuration, a support member 318 is positioned in the cavity 302 to provide additional structural support of the interior surfaces of the flexible enclosure 304 and to control the flow of fluid, such as air, through the acoustic driver 204. The support member 318 may include, for example, rods or baffles. Examples of baffles suitable for this use include segmental baffles, rod baffles, helical baffles, or other such baffles that provide structural support while still allowing fluid flow therebetween. Preferably, the support member 318 is composed of a magnetic resonance imaging compatible material, such as a non-metallic material or a magnetic resonance imaging compatible metal. As illustrated in FIG. 4, the support member 318 can be surrounded by the fill material 314. The support member 318 is coupled to the interior surfaces of the flexible enclosure 304. By way of example, the support member 318 is coupled to the interior surfaces of the enclosure 304 by way of an adhesive, such as double-sided tape or the like.

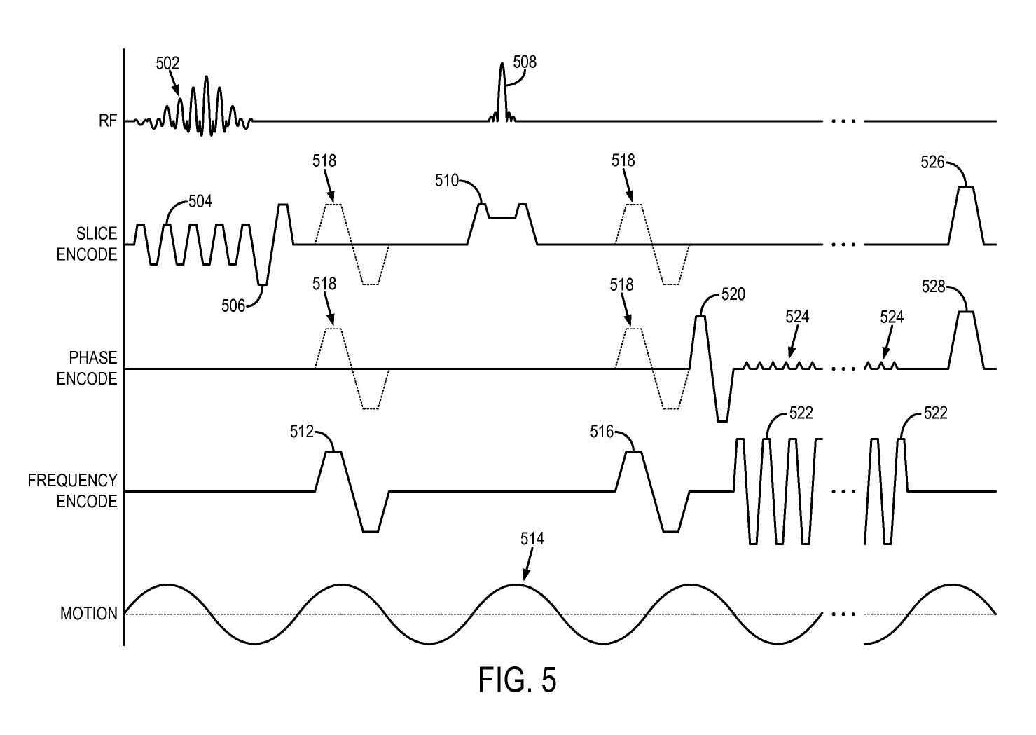

Referring now to FIG. 5, an example of a spin-echo echo-planar imaging (“SE-EPI”) pulse sequence that may be used to acquire MRE data when practicing some embodiments of the present invention is illustrated. The pulse sequence begins with the application of a spatial-spectral radio frequency (“RF”) excitation pulse 502 that is played out in the presence of an alternating slice-selective gradient 504. A spatial-spectral RF excitation is employed to suppress chemical shift artifacts resultant from fat signals; however, it will be appreciated that other excitation schemes can also be employed. To mitigate signal losses resulting from phase dispersions produced by the slice-selective gradient 504, a rephasing lobe 506 is applied after the slice-selective gradient 504.

A refocusing RF pulse 508 is applied in the presence of another slice-selective gradient 510 to induce the formation of a spin-echo. In order to substantially reduce unwanted phase dispersions, crusher gradients bridge the slice-selective gradient 510. A first motion-encoding gradient 512 is played out along a motion-encoding direction before the refocusing RF pulse 508. The frequency of the motion-encoding gradient 512 is set at or near the center frequency of the motion 514 produced by the breast MRE driver. By way of example, this frequency of the motion-encoding gradient 512 may be set at 60 Hz. Following the refocusing RF pulse 508, a second motion-encoding gradient 516 is played out along the motion-encoding direction. For example, as illustrated in FIG. 5, the motion-encoding gradients 512, 516 may be played out along the frequency-encoding direction. In the alternative, as indicted by dashed lines 518, the motion-encoding gradients 512, 516 may be played out along the phase-encoding direction, the slice-encoding direction, or some combination of these three directions so as to encode motion 514 in an oblique direction.

A prephasing gradient 520 is played out along the phase-encoding direction to prepare the transverse magnetization for data acquisition. Then, an alternating readout gradient pulse train 522 is then produced in order to form echo signals from which image data is acquired. For example, gradient-echo signals formed under a spin-echo envelope are acquired during each positive and negative pulse peak of the readout pulse train 522. A phase-encoding gradient “blip” 524 is applied between each readout pulse peak to separately phase encode each acquired gradient-echo signal. Following the conclusion of the readout gradient pulse train 522, a spoiler gradient 526 is played out along the slice-encoding direction and another spoiler gradient 528 is played out along the phase-encoding gradient to prepare the spins for subsequent data acquisitions. The data acquisition is repeated a plurality of times with appropriate changes to the slice selection procedure such that multiple slices of image data are acquired. For breast imaging, spatial saturation bands may be positioned posterior to the breasts to suppress signal from the heart and lungs. Additionally, separate acquisitions may be performed with the RF center frequency on the water and fat resonance peaks.

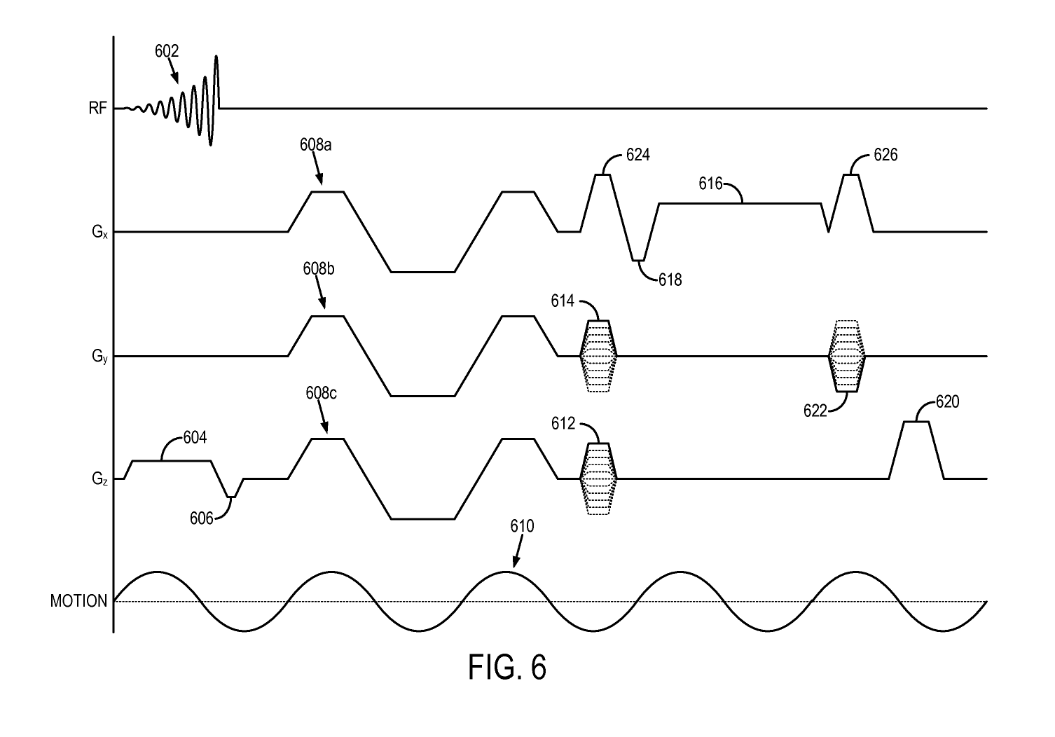

Referring now to FIG. 6, an example of a three-dimensional gradient-recalled echo (“GRE”) pulse sequence that may be used to acquire MRE data when practicing some embodiments of the present invention is illustrated. This pulse sequence is capable of acquiring suitable three-dimensional vector wave field information in three motion axes in both breasts simultaneously. Transverse magnetization is produced by an RF excitation pulse 602 that is played out in the presence of a slice-selective gradient 604. To mitigate signal losses resulting from phase dispersions produced by the slice-selective gradient 604, a rephasing lobe 606 is applied after the slice-selective gradient 604.

Motion-encoding gradients 608a, 608b, 608c are played out along the three gradient axes. These motion-encoding gradients 608 sensitize the transverse magnetization to motion occurring along the direction defined by the motion-encoding gradients 608. The motion-encoding gradients 608 are alternating gradients having a frequency not necessarily equal to that of a drive signal that drives the MRE driver to produce oscillatory motion 610 in the subject. The pulse sequence server 110 produces sync pulses every 4 repetition time (“TR”) periods, during which a total number of 2n+1, n=0, 1, 2, 3, 4, 5, . . . cycles of motion 610 with the desired frequency are applied to the subject. The TR value may be calculated by

$$TR = \frac{( 2 n + 1 ) T}{4} ; \qquad ( 3 )$$

where T is the period of motion 610 and n is non-negative integer, which is selected so that the TR has the minimal required time for performing both the spatial-encoding gradients and the motion-encoding gradients. The duration of the motion-encoding gradients 608 is optimized so that the sequence can have the most motion-encoding sensitivity and smallest echo time. Because of the timing arrangement of TR and the motion 610, four repetition TRs is equal to (2n+1) times the period of the motion 610; thus, the phase of the motion 610 changes by ninety degrees automatically between two neighboring TR periods. This is called quadrature motion sampling.

The phase of the acquired magnetic resonance signals is indicative of the movement of the spins when the motion-encoding gradients 608 are applied. If the spins are stationary, the phase of the magnetic resonance signals is not altered by the motion-encoding gradients 608, whereas spins moving along the motion-encoding direction will accumulate phase proportional to the velocity of the spins' motion. Spins that move in synchronism and in phase with the motion-encoding gradients 608 will accumulate maximum phase of one polarity, and those which move in synchronism, but 180 degrees out of phase with the motion-encoding gradients 608 will accumulate maximum phase of the opposite polarity. The phase of the acquired magnetic resonance signals is, thus, affected by the synchronous movement of spins along the motion-encoding direction.

Phase encoding is performed along two axes: the z-axis and the y-axis. The z-axis, or in-plane, phase-encoding is accomplished by applying a G_{z} phase-encoding gradient 612 and the y-axis phase-encoding is accomplished by applying a G_{y} phase-encoding gradient 614. As is well-known to those skilled in the art, the magnitude of the phase-encoding gradients 612, 614 are stepped through a series of positive and negative values during the scan, but each is set to one value during each repetition of the pulse sequence. It is the order in which these spatial-encoding pulses 612 and 614 are stepped through their set of values that determines the three-dimensional k-space sampling order.

After spatially-encoding the transverse magnetization, the MR signal is read-out in the presence of a Gₓ readout gradient 616. The readout gradient 616 is preceded by a negative gradient lobe 618 to produce a gradient-recalled echo signal in the usual fashion. The readout gradient is bridged by flow compensation gradient 624, which reduces flow-related artifacts. The pulse sequence is then concluded by the application of a large G_{z} spoiler gradient 620, a Gₓ spoiler gradient 626, and a G_{y} rewinder gradient 622 to prepare the magnetization for the next repetition of the pulse sequence. As is known to those skilled in the art, the spoiler gradient 620 dephases transverse magnetization and the rewinder gradient 622 refocuses transverse magnetization along the y-axis in preparation for the next pulse sequence. The rewinder gradient 622 is equal in magnitude, but opposite in polarity with the G_{y} phase-encoding gradient 614.

Image reconstruction and processing of the reconstructed images may also be performed to provide an indication of tissue stiffness as disclosed in U.S. Pat. No. 5,825,186, which is incorporated herein by reference in its entirety. By way of example, when using the pulse sequence illustrated in FIG. 6 to acquire a three-dimensional vector wave field, MRE inversion may be performed by calculating the vector curl of the measured wave data. The vector curl may be calculated using 3×3×3 derivative kernels on the wrapped phase data, as described by K. J. Glaser and R. L. Ehman in “MR Elastography Inversions Without Phase Unwrapping,” Proc. Intl. Soc. Mag. Reson. Med. 17, 2009; 4669. A three-dimensional local frequency estimation (“LFE”) inversion may then be performed in the curl data with two-dimensional directional filtering to produce the MRE elastograms.

The present invention has been described in terms of one or more preferred embodiments, and it should be appreciated that many equivalents, alternatives, variations, and modifications, aside from those expressly stated, are possible and within the scope of the invention.

1. A method for performing magnetic resonance elastography (MRE) of a subject's breast using a magnetic resonance imaging (MRI) system, the steps of the method comprising:
a) positioning an MRE driver on a sternum of a subject;
b) operating the MRE driver so that mechanical energy is imparted to the sternum such that shear waves are produced in at least one breast of the subject;
c) directing the MRI system to acquire image data of the subject while the shear waves are produced in the at least one breast of the subject;
d) reconstructing from the acquired image data, images of the subject that depict propagation of the shear waves through the at least one breast of the subject; and
e) calculating from the reconstructed images, a mechanical property of the at least one breast of the subject.

2. The method as recited in claim 1 in which the MRE driver is positioned in step a) so that it does not contact the at least one breast of the subject.

3. The method as recited in claim 1 in which step c) includes directing the MRI system to apply a spatial-spectral radio frequency (RF) pulse to the at least one breast of the subject before acquiring image data therefrom.

4. The method as recited in claim 1 in which step a) includes positioning a subject in a prone position in a bore of the MRI system.

5. The method as recited in claim 1 in which step a) includes positioning the MRE driver between the subject's sternum and a breast radio frequency (RF) coil.

6. The method as recited in claim 1 in which the MRE driver is positioned in step a) and operated in step b) such that the MRE driver does not add tension to the subject's breast.

7. An acoustic driver for applying acoustic energy to a subject during a magnetic resonance elastography (MRE) examination, the acoustic driver comprising:
a cavity configured to receive acoustic energy;
a flexible enclosure surrounding the cavity, the flexible enclosure being sized for placement adjacent a subject's sternum; and
an intake extending through the flexible enclosure to the cavity and configured to be coupled to a tube to receive acoustic energy for delivery into the cavity.

8. The acoustic driver as recited in claim 7 in which the flexible enclosure is sized for placement adjacent the subject's sternum such that the flexible enclosure does not contact the subject's breasts.

9. The acoustic driver as recited in claim 7 in which the flexible enclosure includes a flexible membrane configured to be placed into contact with the subject's skin.

10. The acoustic driver as recited in claim 9 in which the flexible membrane is composed of at least one of woven fabric, polycarbonate plastic, polystyrene foam, foam rubber, and a non-stretching material mesh.

11. The acoustic driver as recited in claim 9 in which the flexible enclosure further includes a wall opposing the flexible membrane and side walls extending from the wall to the flexible membrane such that the cavity is defined therebetween.

12. The acoustic driver as recited in claim 7 further comprising a porous fill material that substantially fills the cavity.

13. The acoustic driver as recited in claim 12 in which the fill material is at least one of a polyfiber material and woven fabric.

14. The acoustic driver as recited in claim 7 further comprising a support member disposed within the cavity and configured to support interior surfaces of the flexible enclosure.

15. The acoustic driver as recited in claim 14 in which the support member is composed of a magnetic resonance imaging compatible material.

16. The acoustic driver as recited in claim 14 in which the support member includes at least one of rods and baffles.

17. The acoustic driver as recited in claim 14 in which the support member is coupled to the interior surfaces of the flexible enclosure.

18. The acoustic driver as recited in claim 7 in which further comprising a disposable cover disposed about the flexible enclosure.

19. The acoustic driver as recited in claim 18 in which the disposable cover is at least one of a cloth and a film.

20. The acoustic driver as recited in claim 18 in which the acoustic driver is sterilized and configured to be disposable after use.
